SEQUENCE LISTING (6 sequences)

SEQ ID NO: 1 - DNA, 19 bp, Artificial Sequence
  Description of Artificial Sequence Synthetic primer
tggctactaccgaagagct

SEQ ID NO: 2 - DNA, 20 bp, Artificial Sequence
  Description of Artificial Sequence Synthetic primer
tgcagcattgttagcaggat

SEQ ID NO: 3 - DNA, 41 bp, Artificial Sequence
  Description of Artificial Sequence Synthetic primer
tctggcccagttcctaggtagtccagacgaattcgtggtgg

SEQ ID NO: 4 - DNA, 40 bp, Artificial Sequence
  Description of Artificial Sequence Synthetic primer
agacggcatcatatgggttgcacgggtgccaatgtgatct

SEQ ID NO: 5 - DNA, 25 bp, Artificial Sequence
  Description of Artificial Sequence Synthetic primer
ggactgagatctttcattttaccgt

SEQ ID NO: 6 - DNA, 21 bp, Artificial Sequence
  Description of Artificial Sequence Synthetic primer
actgagggagccttgaataca

(19) United States
(12) Patent Application Publication
(10) Pub. No.: US 2023/0075234 A1
(43) Pub. Date: Mar. 9, 2023

(54) Composite Energetic Material With Self-Regulated Temperature

(71) Applicant: The Trustees of the University of Pennsylvania, Philadelphia, PA (US)

(72) Inventors: Haim H. Bau, Swarthmore, PA (US); Michael G. Mauk, Philadelphia, PA (US); Ruijie Li, Wexford, PA (US)

(21) Appl. No.: 17/738,480

(22) Filed: May 6, 2022

Related U.S. Application Data
(60) Provisional application No. 63/185,569, filed on May 7, 2021.

(51) Int. Cl. B01L 3/00; F24V 30/00; F28D 20/00; B01L 7/00; C12Q 1/70

(52) U.S. Cl. CPC B01L 3/50851; F24V 30/00; F28D 20/0056; B01L 7/52; C12Q 1/701; F28D 2020/006; B01L 2300/1805; B01L 2300/0627; C12Q 1/6844

(57) ABSTRACT

An exothermic composite, comprising: a reactive material (RM) that undergoes an exothermic reaction upon contact with an oxidizer, and a phase-changing thermal storage material (PCM) having a phase change temperature, wherein (1) RM and PCM are intermixed with one another or (2) one of RM and PCM is interpenetrated with the other. Devices, comprising (1) a sample container that defines a sample volume therein or (2) a receptacle configured to accept a sample container defining a sample volume therein, and the device configured such that the sample container is in thermal communication with a composite according to the present disclosure. Also provided are related methods.

### CROSS-REFERENCE TO RELATED APPLICATIONS

This application claims priority to and the benefit of U.S. Provisional Application No. 63/185,569, “Composite Energetic Material With Self-Regulated Temperature” (filed May 7, 2021), the entirety of which foregoing application is incorporated by reference herein for any and all purposes.

### GOVERNMENT RIGHTS

This invention was made with government support under AI134594 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Nov. 7, 2022, is named 103241_ 006834_21-9500_SL.txt and is 1,677 bytes in size.

### TECHNICAL FIELD

The present disclosure relates to the field of materials and methods for electricity-free heating and temperature control and to the fields of thermally-modulated amplification reactions and point-of-care diagnostics.

### BACKGROUND

Short of widely available vaccines and/or herd immunity, effective control of pandemics such as COVID-19 requires frequent, extensive screening with prompt results. Epidemiology models recommend testing individuals as often as 2-3 times a week to inform these individuals whether they should self-quarantine or may go to work or school without endangering others; to enable contact tracing; and to enable policy makers adapt informed, rational, region-specific control measures.

Strategies based on self-collection of samples and mailing these samples to centralized laboratories for testing involve significant delays between sample collection and results, defeating one's ability to take prompt preventive measures after exposure. Although hand delivery of samples to centralized collection sites eliminates mailing time, the process still takes 1-2 days to obtain results; carries some risk of exposure to the sample provider and others; and is inconvenient. Optimally, tests should be carried out at home, enabling a test and isolate strategy without the need to travel and que at clinics or at sample collection sites; thereby minimizing the risk of contracting/spreading the disease as well as reducing inconvenience, lost time from work, and lost productivity.

Currently, ‘molecular’ diagnostics (enzymatic Nucleic Acid Amplification Tests, NAATs) are the most sensitive and specific tests for pathogen detection. The development of portable, easy-to-use, low-cost medical diagnostics technology has been the focus of substantial efforts for three decades and is especially critical during the current COVID-19 pandemic. Although the polymerase chain reaction (PCR) is the gold standard of molecular diagnostics, PCR is challenging to implement at the point-of-care (POC) because it requires precise thermal cycling, stringent sample preparation, and either optical or lateral flow-based detection. The advent of isothermal nucleic acid amplification has simplified sample processing and thermal control requirements, making inexpensive point-of-care (POC) and home molecular tests possible.

Reverse Transcription Loop mediated amplification (RT-LAMP) is an isothermal enzymatic amplification method that has gained wide acceptability during the COVID-19 epidemic. RT-LAMP requires a constant incubation temperature, ranging from 60 to 65° C., for about 30 minutes. RT-LAMP is more tolerant of contaminants and requires less stringent sample preparation than PCR. The RT-LAMP process also produces about an order of magnitude more amplicons than PCR. The abundance of RT-LAMP amplicons enables a plethora of detection methods, ranging from fluorescent intercalating dye, fluorescent probes, colorimetric dyes, bioluminescence, and turbidity. Colorimetric dyes can be detected by eye without a need for a reader.

Although the temperature control requirements of RT-LAMP are much simpler than that of PCR, the process still needs to be carried out at a fixed temperature, which requires an incubator. Generally, such incubators consist of an electrical heater and a thermal controller; may represent a significant cost for home use; require substantial fabrication time; and are susceptible to supply line shortages such as the current global shortage in semiconductor chips, which may prevent rapid deployment. Further, some chemical and biological processes require multi-step heating, e.g., heating that is maintained at a first temperature for a first period of time and heating that is maintained at a second temperature for a second period of time. Accordingly, there is a long-felt need in the field for improved materials and methods for controlled heating (including multi-step heating) in chemical and biological processes, including, e.g., RT-LAMP and other reactions.

### SUMMARY

In meeting these long-felt needs, the present disclosure provides an exothermic composite, comprising: a reactive material (RM) that undergoes an exothermic reaction upon contact with an oxidizer, and a phase-changing thermal storage material (PCM) having a phase change temperature, wherein (1) RM and PCM are intermixed with one another or (2) one of RM and PCM is interpenetrated with the other.

Also provided are devices, comprising (1) a sample container that defines a sample volume therein or (2) a receptacle configured to accept a sample container defining a sample volume therein, and the device configured such that the sample container is in thermal communication with a composite according to the present disclosure (e.g., any one of Aspects 1-16).

Further provided are methods, comprising: contacting, with an oxidant (also termed an oxidizer), an exothermic composite according to the present disclosure (e.g., any one of Aspects 1-12), the contacting giving rise to a sample container that is in thermal communication with the composite maintaining a temperature of about the phase change temperature of PCMA, and effecting a biological or chemical interaction in the sample container.

Additionally provided are garments, shelters, blankets, or containers comprising a composite according to the present disclosure (e.g., any one of Aspects 1-16).

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1A-1B: Chemical heater-based, instrumentation-free, molecular detection of SARS-Cov-2 and other pathogens in saliva. (FIG. 1A) Test workflow: (1) collect saliva sample in a tube; (2) mix saliva with lysis buffer; (3) aliquot the sample to individual tubes, each dry-storing RT-LAMP reaction mix specific to the selected target; (4) insert tube(s) in the chemical heater and add water to the EPCM to initiate exothermic reaction and heat the tubes to 60-65° C. for ˜30 minutes. (FIG. 1B) Color change of the tube indicates whether the test is positive (yellow, three tubes) or negative (red, one tube). Here, the negative test is a negative control to verify that the color change does not occur because of the chemical composition of the sample.

FIGS. 2A-2C: The tube temperature as a function of time (FIG. 2A) when the ambient temperature is 10° C., 22° C., and 40° C. (1.4 g Mg(Fe), 6 mL water, all experiments in triplicate); (FIG. 2B) when the water volume is 4, 6, and 8 mL (1.4 g Mg(Fe), ambient temperature 22° C.); and (FIG. 2C) when the Mg(Fe) mass is 1.05 g and 1.4 g (6 mL water, ambient temperature 22° C.). In all cases, the mass of the PCM (PureTemp 63) is 10.5 g.

FIG. 3: Real-time monitoring of RT-LAMP incubated with a chemical heater. (Left panel) schematics of experimental set up to monitor fluorescence intensity of the RT-LAMP reaction, amplifying COVID-19 RNA in diluted saliva. (Right panel) Fluorescence (EvaGreen™ intercalating dye) emission captured with our USB microscope (see FIG. 5 for a photograph) as a function of time from positive samples. The insets are photographs of the fluorescent signal detected with the USB microscope through the lid of the test tube.

FIG. 4. The magnesium alloy and PCM before (left) and after (right) mixing.

FIG. 5. Chemical heater with real time fluorescent monitoring.

FIG. 6. Chemical heater device: (bottom) cup, (middle) lid, (top) lid and cup assembled.

FIG. 7. Amplicon detection with LCV. (left) positive test. (right) negative test

FIG. 8. Benchtop amplification curves (fluorescent mission intensity as a function of time, min) for the same samples that were tested with our chemical heater. Samples diluted ten-fold and hundred-fold produce amplification curves. The sample diluted thousand-fold does not present an amplification curve, suggesting that the benchtop instrument and the chemical heater have similar limits of detection.

FIG. 9. Depiction of exemplary device configured to effect two-stage heating; the device is configured to contain two different phase change materials.

FIG. 10. Depiction of a two-stage heater, showing the outer cup (to be filled with composite material including fuel and a phase change material having a comparatively high phase change temperature) and the inner cup (to be filled with a phase change material having a comparatively low phase change temperature).

FIG. 11. Provides exemplary schematics of temperature vs. time graph for a two-stage heating according to the present disclosure.

FIG. 12. Provides example temperature vs. time profiles for a Mg/Fe+H₂O composition and an EPCM (Energetic Phase Change Material) that includes Mg/Fe, H₂O, and a phase change material (PCM).

FIG. 13. provides an example device according to the present disclosure, which device includes a microfluidic chip in thermal communication with (and contacting) an EPCM, with thermally insulating container disposed around the EPCM. In this arrangement, the top surface of the microfluidic chip is exposed for fluidic exchange and optical excitation and interrogation. The configuration shown can accommodate modifications, e.g., including water pouches and/or capillary channels to distribute water to initiate and sustain the exothermic reaction.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

### Exemplary Disclosure

The following exemplary disclosure illustrates a particular embodiment of the disclosed technology and does not limit the scope of the present disclosure or the appended claims.

Molecular detection of pathogenic nucleic acids from patient samples requires incubating biochemical reactions at specific temperatures to amplify DNA. This incubation is typically carried out with an electrical heater and a temperature controller. To reduce test cost; eliminate the need to manufacture incubators, which may require significant time; and to enable electricity-free operation, we use an energetic compound such as Mg(Fe) alloy mixed with a phase-change material (PCM) that undergoes phase transformation at the desired incubation temperature. In the present disclosure, we term this composite Energetic Phase Change Material (EPCM). When the EPCM is brought into contact with water, the magnesium alloy interacts with the water to produce heat. The EPCM heats up to its phase transition temperature. Any excess heat is absorbed as latent heat and the system is maintained at its desired incubation temperature, independent of ambient temperatures, long enough to facilitate polymerase. The EPCM together with colorimetric amplicon detection facilitates inexpensive, disposable point-of-need diagnostic test that does not require any electric power. We demonstrate the feasibility of our approach by detecting SARS-Cov-2 in saliva samples either without any instrumentation or with a CCD camera that enables us to follow the amplification process in real time.

Incubator

To obtain combined heating and temperature control, we mix for 5 minutes (FISHER brand® Vortex Genie 2™12-812) magnesium-iron (Mg/Fe) powder like the one used in Meals-Ready-to-Eat (flameless rations for military and outdoor recreation food cooking) (MRE, US 1992 06421, 8970-01-321-9153) with PureTemp-63 PCM in granular form (PureTemp, Minneapolis) to form a nearly homogeneous composite mixture, dubbed EPCM (Energetic Phase Change Material). The bio-based PureTemp™ materials are nontoxic and can be synthesized to provide any desired phase change temperature in the range from −37° C. to 151° C. PureTemp 63 undergoes phase transition from solid to liquid in the temperature range between 61° C. and 65° C. and has latent heat of 208 kJ/kg.

Addition of water to the magnesium alloy triggers the exothermic reaction:

$$Mg ( Fe ) ( s ) + 2 {H}_{2} O \to {Mg ( OH )}_{2} ( s ) + {H}_{2} ( g ) - 353 \frac{k J}{mol Mg} ( 1.41 {0}^{7} \frac{k J}{kg - Mg} ) \qquad ( 1 )$$

with substantial release of energy. Initially, the released energy increases the system's temperature until it reaches the phase transition temperature. Thereafter, any further energy release from the reaction (1) effectuates phase transformation and is stored as latent heat in the PCM without further increasing its temperature. This reaction, which may be electrochemical in nature, can be accelerated by the inclusion of small amounts of salts (such as NaCl). The salt can be premixed with the Mg(Fe) powder in variable proportions.

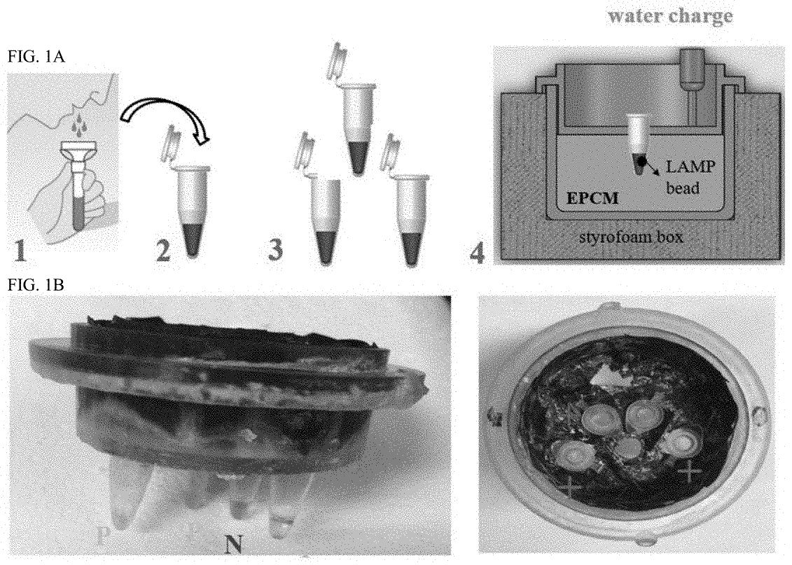

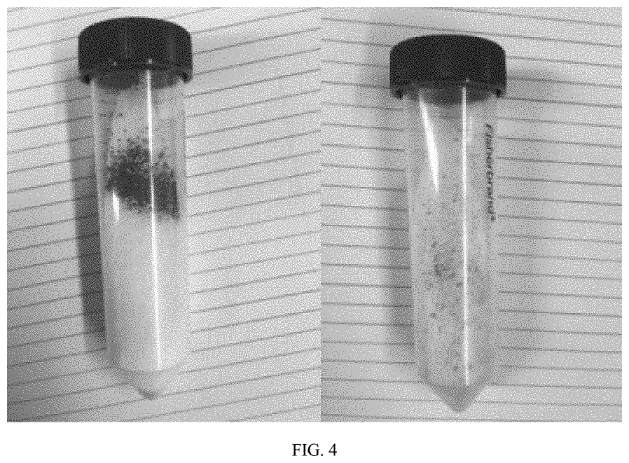

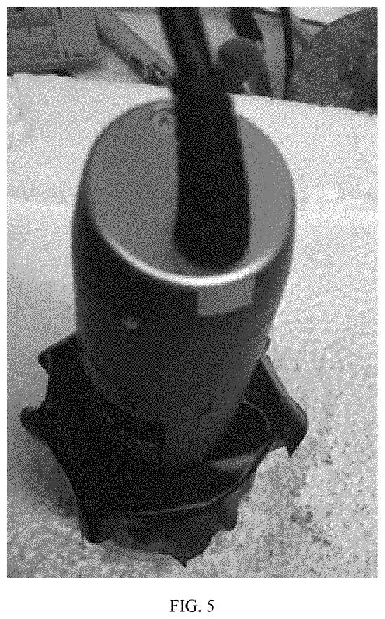

We placed our EPCM mixture in a 35 mm diameter, 25 mm tall, custom made, 3D-printed cup with 2 mm thick wall (FIG. 4). The cup was, printed with photopolymer clear resin on a Form 3 stereolithographic printer (Formlab, Somerville, Mass.). The cup's lid (FIG. 5 center) includes a few bores to accommodate 200 μL tubes (BIO-RAD, #TFI0201). Three 1-mm wide slots around the lid's periphery allow the venting of any hydrogen gas produced during the exothermic reaction (Eq. 1). The cup is placed in a Styrofoam block for thermal insulation. In operation, about ⅔ of each tube's length is submersed in the EPCM. We coated the top of the lid with black electrical tape or 2-mm thick black foam rubber to suppress background fluorescence reflection/emission (FIG. 1B). Such a coating is not needed for colorimetric detection. To further reduce cost, one can replace the 3D-printed cup with molded styrofoam.

The cup can accommodate multiple tubes. These tubes can house dry-stored reaction mixes and primers, with each tube specialized to detect a specific target pathogen of interest. One of these tubes operates as a negative control and another tube can be used as a positive control to detect targets that are present in all samples, e.g., beta actin. The positive control verifies the reagents integrity; the absence of inhibitors in the sample; and the appropriateness of the incubation temperature.

After the insertion of the sample into the tubes, the tubes are placed into the cup and water is added to the EPCM from above to initiate the exothermic reaction. Since the water only partially wets the PCM, it takes 2-3 minutes for the water to infiltrate into the porous EPCM. Some of the water may absorb into the PureTemp™ and over time degrade the PCM. This is, however, not a concern here since we use the biodegradable PCM for a relatively short time and dispose of it after use.

Once water is added to the cup, the water seeps through the EPCM and reacts with the Mg(Fe) alloy. The, hydrogen gas produced during this reaction percolates up through the powder mix. After the EPCM has reached the PCM's phase change temperature, any additional energy produced by the reaction is consumed as latent heat—transforming the PCM from a solid into a liquid—instead of increasing the system's temperature; thereby, our system maintains its desired incubation temperature independent of the ambient temperature within a broad range of ambient temperatures. After the chemical reaction ran its course, thermal losses to the environment are mitigated by the release of latent heat as the PCM transforms back from a liquid into a solid, enabling the reaction chamber to maintain a nearly uniform temperature for time intervals well beyond what is needed for the amplification process.

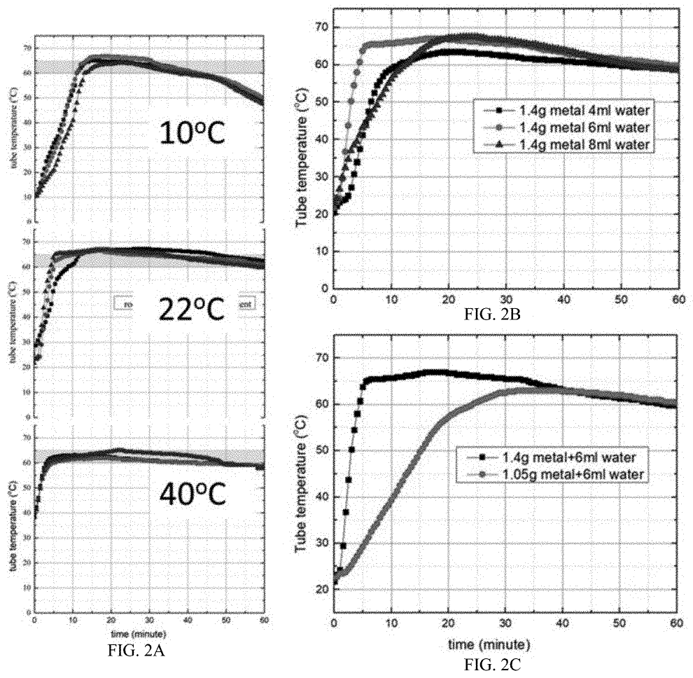

To characterize our system's thermal performance, we placed a thermocouple (OMEGA, K type, #TFIR-245-50) into one of the tubes in the presence of 20 μL of water and monitored the tube's temperature as a function of time. These experiments were carried out in an environmental oven with the oven temperature ranging from 10° C. to 40° C. (FIG. 2A) to mimic operation under various ambient conditions. Despite the significant variations in the ambient temperature, our EPCM maintains a reasonably stable incubation temperature as needed for the RT-LAMP process. We performed all experiments in triplicate, with good reproducibility.

To minimize the temperature ramp up time, we used 6 mL of water (4.3 mL water per gram of Mg(Fe)), which exceeds the stoichiometric water mass (2.1 mL). The use of excess water increases the rate of water infiltration into the EPCM, reducing the temperature ramp up time, and making up for any water absorbed by the PCM and for water evaporation. Smaller (4 mL) and greater (8 mL) volumes of water increased the temperature ramp up time from approximately 3 min to nearly 10 min (FIG. 2B) without a significant change in the incubation temperature. Hence, it appears that 6 mL water is nearly optimal for our set-up. The ramp up time also depends on the mass ratio of the Mg(Fe) fuel and the PCM. A reduction in the Mg(Fe) mass results in an increase in the ramp up time (FIG. 2C).

Molecular Diagnostics

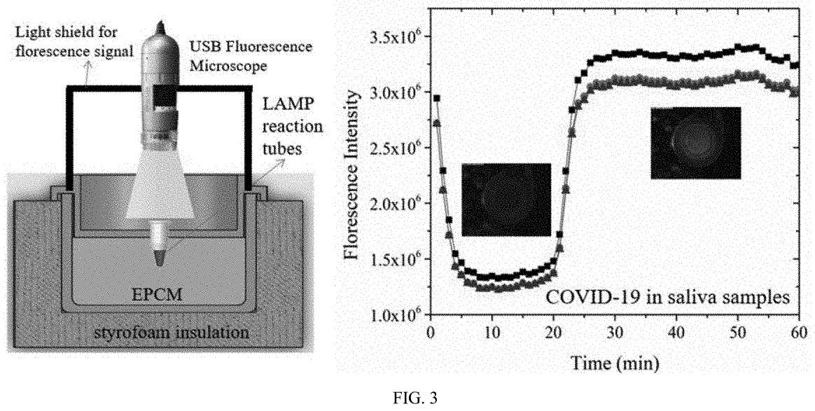

To assess the diagnostic capabilities of our system, we used contrived samples of SARS-Cov-2 virions isolated from a cell line and suspended in saliva donated by healthy individuals. We incubated our samples at 65° C. for 15 min to lyse and inactivate the virus. The samples were then diluted 10, 100, and 1000-fold. The saliva samples were aliquoted into three 200 μL tubes together with the LAMP reaction mix (WarmStart® Colorimetric LAMP 2X Master Mix, New England Biolabs, USA augmented with EvaGreen fluorescent dye). In each case, we added a set of six primers targeting the N3 gene of SARS-Cov-2 to a total reaction volume of 20 μL per tube. We triplicated our test to examine reproducibility. In applications, each tube may contain a reaction mix for a different target. The fourth tube was used as a negative (no primer) control to examine for possible color change resulting from sample composition. Once the tubes were sealed, they were placed into our heating cup (FIG. 1A and FIG. 1B) and water was added to the EPCM to initiate the exothermic reaction. We used two methods to detect the presence of amplicons: colorimetric dye (FIG. 1B) and fluorescent intercalating dye (FIG. 3). Concurrently, the same samples were processed with a benchtop (BIO-RAD CFX96′ Real-Time System, C1000 Touch Thermal Cycler) thermal cycler programmed to operate at a fixed temperature (65° C.).

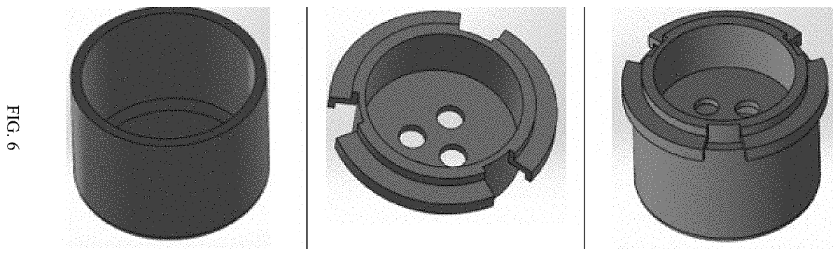

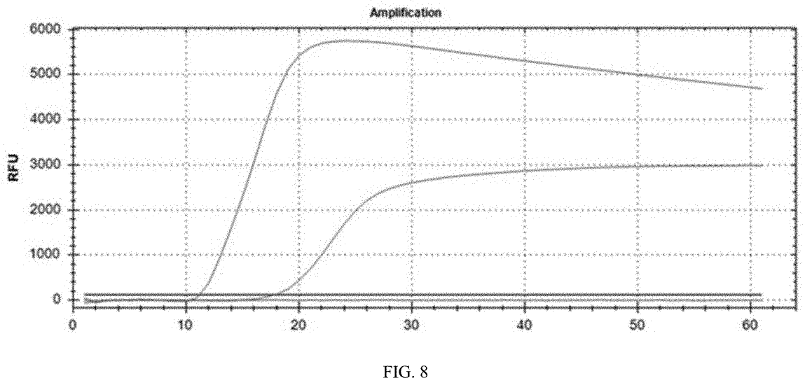

The colorimetric detection in FIG. 1 uses the pH indicator phenol red. Since one of the polymerase reaction byproducts is protons, the amplification process leads to a decrease in the pH of the reaction mix. This change in pH can be readily observed with pH indicators. In our case, the phenol dye changes color from red to yellow when polymerase takes place (FIG. 1B). The pH indicator is not optimal for testing saliva because the saliva's pH may vary among individuals and may depend on diet. In our assay, the negative control guards against false positives such as change of color resulting from sample acidity. A color change of the negative control would invalidate our test. Alternatively, we can use other colorimetric dyes that are insensitive to pH such as the intercalating dye Luco Crystal Violet (LCV) that changes from colorless to violet in the presence of dsDNA as we have previously described. See FIG. 6. The color change can be observed either through the tube lid (FIG. 1B right)) or by removing the cup's lid at a prescribed time (e.g., 30 min) after the start of the heating process (FIG. 1B, left). In our experiments, we detected no false positives. All the tubes containing templates changed color, indicating a reproducible positive test while the negative control did not change color (FIG. 1B). Both our tests with the EPCM and the benchtop (SI FIG. 8) yielded equivalent results. We obtained positive results with ×10 and ×100-fold diluted samples but failed to produce a positive signal with a ×1000-fold dilution. Our instrumentation-free home test appears to have a similar sensitivity to that of the benchtop RT-LAMP.

We can also monitor the amplification reaction in real time with intercalating dye (EvaGreen™). This dye is quenched while in solution emitting relatively low intensity fluorescence. In the presence of dsDNA, the dye binds to the dsDNA and its emission intensity increases greatly. We excited and detected the fluorescent dye with a portable CCD camera (Dino-Lite Edge AM4115T.GFBW-R9) (FIG. 3). At low temperatures (prior to amplification), we observe significant emission from the dye possibly due to the presence of primer target complexes. As the temperature increases, the emission intensity decreases both due to reduced emission efficiency with temperature (as is common to most fluorescent dyes) and the dissociation of short double stranded fragments. Once amplificons are produced, the emission intensity increases. Consistent with the benchtop experiments and the colorimetric detection, tests with ×10 and ×100-fold sample dilutions yielded positive results while the ×1000 dilution did not yield a positive signal.

### DISCUSSION

We have demonstrated the feasibility of incubating RT-LAMP reaction instrumentation and electricity—free with the reaction temperature being self-regulated through the use of the energetic composite comprised of the PCM PureTemp63 and the reactive material Mg(Fe). Our incubator is made of readily available, inexpensive materials, enabling fast deployment. Since we use just a few grams of Mg(Fe) and PureTemp, the materials cost just a few pennies. Our approach has a few distinguishing features compared to previous implementations of chemical heaters. By mixing the reactants and the PCM, we minimize heat transfer resistance between the exothermic reaction and the reaction tube without a need for thermal waveguides or enhancement of PCM conductivity with dispersed high conductivity particles as was previously done. Our EPCM enables our system to go from room temperature to the RT-LAMP operating temperature within 3 minutes. The reaction rate in our system is tempered because of the slow infiltration of the water into the powder mix, which prolongs the reaction and prevents an initial significant temperature overshoot that may denature LAMP enzymes. The porous nature of the EPCM also allows for the escape of hydrogen gas product without forming hydrogen bubbles and/or splattering of reactants. Since PCMs are available with a wide range of phase transition temperatures (e.g., ranging from −37 to +151° C.), the method described herein could be applied to various incubation reactions including other isothermal amplifications, ligations, and restrictions.

Our ability to insert test tubes directly into the EPCM simplifies hardware design and manufacturing. In our experiments, we packaged the EPCM in a 3D-printed plastic cup, which is inserted into a Styrofoam block for thermal insulation. The plastic cup can be replaced, however, with other readily available materials such as Styrofoam to further reduce cost.

Since the RT-LAMP process produces a great number of amplicons, it provides diverse opportunities for detecting amplification results, ranging from fluorescent dyes like in PCR to colorimetric dyes. Here, we show results obtained with pH indicator that changes color from red to yellow in positive tests. Alternatively, we can use, among other things, LCV dye. The ability to incubate the reaction without an electrical system and to observe test results without a reader, provides us with a very inexpensive system that may rival in cost lateral flow-based rapid tests while maintaining the advantages of molecular tests such as high specificity and sensitivity and rapid adaptability to new targets.

Our EPCM incubator provides nearly equivalent performance to that of the state-of-the-art benchtop machine. Samples that were positive in the benchtop test were also positive when incubated with our EPCM, and samples that were negative on the benchtop were also negative with our disposable test. Since we do not know the viral load of our samples, we cannot report on limits of detection. However, the limit of detection depends not only on incubation conditions, but to a greater degree on the assay, the development of which is not part of this project. Based on prior work, we anticipate that a limit of detection of 10 target copies per reaction is achievable.

When it is desirable to monitor the amplification reaction in real time, one can use a USB-CCD camera placed a few centimeters above the tube(s) lid to monitor fluorescence emission from DNA-intercalating dye or molecular probes included in the reaction mix. Instead of a dedicated camera, one can use a smartphone, wherein the fluorescence is excited with the smartphone flash and detected with the smartphone camera as we have previously demonstrated.

Here, we have demonstrated our system's capability when operating with saliva samples and testing for COVID-19. Our system can operate, however, with other types of samples such as urine, blood, and food. While the sample preparation workflow depends on the sample type and the assay, the incubation process is generic.

Materials

Mg/Fe powder was obtained in the form of Flameless Ration Heaters (also called self-heating MREs, Meals-Ready-To-Eat) packets. The metal powder is comprised of Mg particles, ranging in diameter from 0.3 mm to 0.5 mm mechanically alloyed with smaller particulates of Fe, and mixed with ˜1% NaCl by mass. This formulation reflects the electrochemical nature of the aqueous exothermic reaction between the magnesium anode and atmospheric oxygen cathode. The iron protects the magnesium against corrosion. The NaCl increases the electrolyte conductivity and has been reported to significantly affect reaction kinetics. Since we obtain the powder in small quantities, it is difficult to assess the cost of the powder purchased in bulk, but a conservative estimate is much less than $0.10 per gram.

Many types of materials in powder form can function as a phase change material (PCM) with desired phase transition temperatures. These include paraffin waxes of various molecular masses, polyethylene glycols, fatty acids such as palmitic acid, and various proprietary PCMs. Here we use the granular, wax-like PureTemp™ 63 (PureTemp, previously Entropy Solutions, Inc., (Minneapolis, Minn.) that undergoes phase transition in the temperature range between 61° C. and 65° C. and has, latent heat of 206 J/g, a solid specific mass of 0.84 g/cm³, and is comprised of roughly spherical particles varying in diameter from 0.4 to 0.6 mm. This material is derived from agricultural products, is biodegradable and non-toxic, and costs in small lots approximately $0.025 per gram.

We place weighted amounts of the Mg/Fe powder (0.45 grams) and PCM material (10.5 grams) in a 5 ml tube, and thoroughly mix for a few minutes (FISHER brand® Vortex Genie 2™12-812) to form a homogeneous mixture with approximately 20 mL total (FIG. 4).

### Experimental Set-Up

To determine time-temperature heating curves, a 200-μl tube was filled with 30 microliters of water and inserted into the chemical heater. A type-K thermocouple (0.5-mm diameter, plastic coated) is inserted through a hole drilled in the tube's lid, and then sealed with acrylonitrile cement, assuring that the thermocouple remains submerged in the water. A second thermocouple is inserted in the powered Mg/Fe-PC material mixture, to directly monitor the EPCM temperature. This latter measurement depends on the position (depth) of the thermocouple in the powder. Both temperatures were recorded every 30 seconds with an Arduino MEGA 2560 microcontrollers and MAX 31856 thermocouple module; sent through the microcontroller serial port to a PC with Parallax™ PLX DAQ data acquisition software; and saved into a Microsoft Excel™ spreadsheet. A few of the experiments were carried out in an environmental chamber (Benchmark myTemp™ digital incubator, Model H2200-HC) to assess the effects of various ambient temperatures (10 to 40° C.) on system's performance.

The real-time fluorescence emission was detected with a Dino-Lite (AM411ST-GFBW) USB digital fluorescent microscope with built-in 375-nm UV LEDs for exciting fluorescence and a 510-nm filter to detect the green fluorescence from DNA-intercalating dyes added to the reaction mixture (FIG. 4). The 1.3 Mega (1280×1024) pixel images were captured with a CMOS camera and saved in JPG format. An image was captured every 30 seconds. The captured images were processed with the MATLAB™ image processing software. Several green pixel intensities were selected and averaged for each capture time to generate a fluorescence intensity vs time curve.

LAMP Reaction and Dye Materials and Protocol

A LAMP reaction of 20 μl total volume comprised:

10 μl WarmStart® Colorimetric LAMP 2X Master Mix (for DNA and RNA) from New England Biolabs (Ipswich, Mass., USA)

Set of Six primers for amplifying COVID-19 gene N3:

| SEQ ID | Primer | | Concentration |
|---|---|---|---|
| NO: | name | Sequence (5′ to 3′) | (μM) |
| 1 | F3 | TGGCTACTACCGAAGAGCT | 0.2 |
| 2 | B3 | TGCAGCATTGTTAGCAGGAT | 0.2 |
| 3 | FIP | TCTGGCCCAGTTCCTAGGTAG | 1.6 |
| | | TCCAGACGAATTCGTGGTGG | |
| 4 | BIP | AGACGGCATCATATGGGTTGC | 1.6 |
| | | ACGGGTGCCAATGTGATCT | |
| 5 | Loop F | GGACTGAGATCTTTCATTTTA | 0.8 |
| | | CCGT | |
| 6 | Loop B | ACTGAGGGAGCCTTGAATACA | 0.8 |

0.8 μl Evagreen® green, fluorescent nucleic acid binding dye (20× dilution) (Biotium, Freemont, Calif., USA).

Target: 1.4 μl isolated COVID-19 RNA diluted 100× in human saliva

H₂O 6.8 μl

The reaction time was 30 minutes for end-point detection.

Detection of Reaction Products with LCV Dye

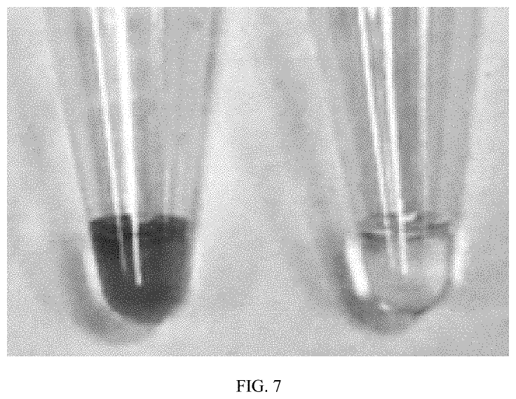

A mixture of Leuco crystal violet (LCV) with sodium sulfite is added to the reaction mix. In the absence of amplicons, the compound LCV-sodium sulfite is nearly colorless. In the presence of dsDNA, LCV binds to the double-stranded DNA and changes from colorless to violet. Therefore, positive test is indicated by color change from colorless to violet (FIG. 7).

### Additional Embodiments

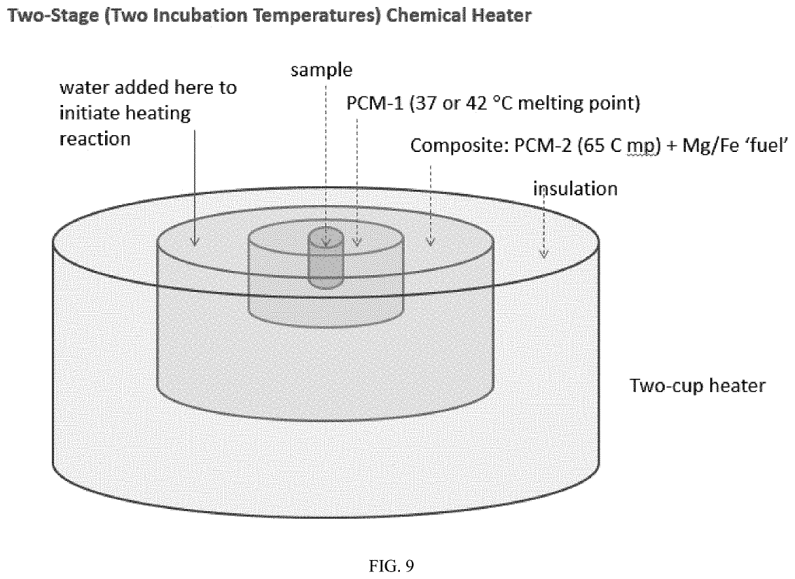

An exemplary device that comprises two different phase change materials is shown in FIG. 9. As shown, such a device can be constructed as two cups or chambers, in which the outer chamber has disposed therein a composite material that includes a second phase change material (PCM-2) and a material (e.g., Mg/Fe alloy) that undergoes an exothermic reaction when contacted with moisture. The inner chamber can have disposed therein a first phase change material (PCM-1). As shown, the second phase change material can have a melting temperature that is higher than the melting temperature of the first phase change material. The device can be configured such that a sample is disposed within the region of the first phase change material. The device can also include an amount of thermal insulation (e.g., foamed polystyrene) so as to thermally insulate the composite material.

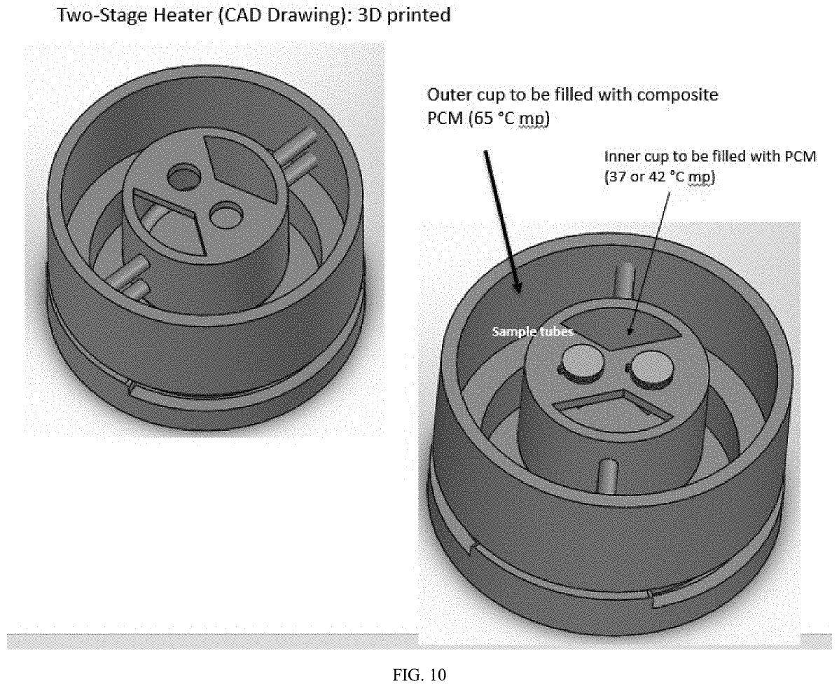

An example two-chamber component is provided in FIG. 10. As shown, such a device can include an outer cup (configured to contain the composite heating material) and an inner cup (configured to contain the second phase change material).

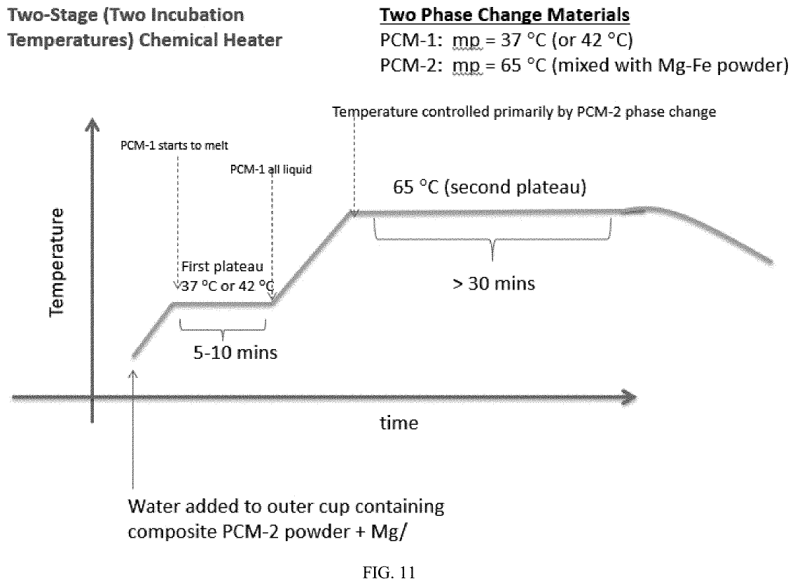

FIG. 11 provides schematics of an exemplary temperature vs. time plot for a device that comprises two different phase change materials, e.g., a device according to FIG. 9 and/or FIG. 10. As shown in FIG. 11, water is initially contacted to the outer cup that contains within it the composite material, which composite comprises PCM-2 (having a melting temperature of about 65 deg C.) and a moisture-reactive Mg/Fe alloy. As the composite material heats up, PCM-1 (disposed in the inner cup, and having a melting temperature of about 37 deg. C or about 42 deg. C) starts to melt. As PCM-1 undergoes the melting phase change, the temperature of the sample reaches the melting temperature of PCM-1 and then plateaus for a period of time (e.g., about 5-10 minutes). Once PCM-1 melts and becomes liquid, the temperature change is then primarily controlled by the PCM-2 phase change material (which has a higher melting temperature than PCM-1), and the sample temperature rises until that sample temperature reaches and then plateaus for a period of time (e.g., 30 minutes or longer) at the melting temperature of PCM-2. The temperature of the sample eventually falls off, as shown by the right-hand side of the plot. By selecting, inter alia, the relative amounts of the moisture sensitive material, and the amount and type of the phase change materials (here, PCM-1 and PCM-2), the user can control the duration of the temperature plateaus. In this way, the user can effect temperature plateaus (of a temperature and duration) that are sufficient to support a two-stage reaction, e.g., RT-LAMP or other reactions.

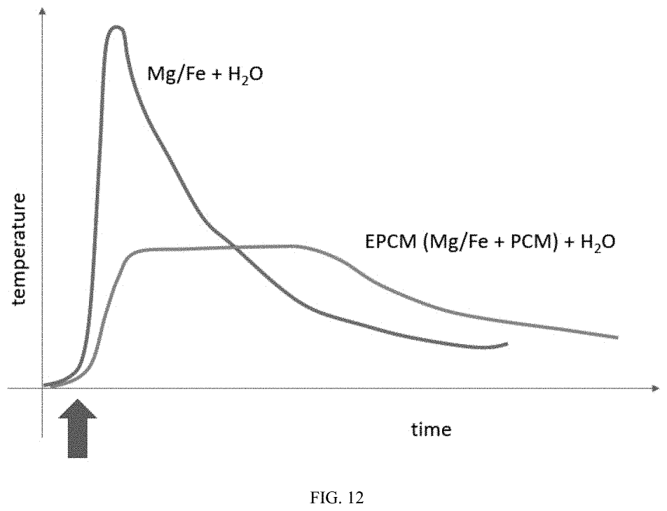

FIG. 12 provides example temperature vs. time profiles for a Mg/Fe+H₂O composition and an EPCM (Energetic Phase Change Material) that includes Mg/Fe, H₂O, and a phase change material (PCM), wherein the phase change material is used to moderate the rate of the temperature rise and the magnitude of the maximum temperature.

There are many instances where comparatively brief heating steps are beneficial for deactivating pathogens, lysing components of samples, releasing reagents encapsulated with temperature-sensitive coatings, activating phase change ‘wax’ valves, and heating expanding beads such as Expancel™ beads to actuate and control liquid flow. The use of magnesium-iron-salt granules and water alone results in rapid heating steps that are difficult to tailor with regard to maximum temperature and duration of heating. These reactions can be vigorous, tending to overshoot the desired upper temperature limits, generate large hydrogen bubbles and splatter material; and are generally undesirable, not reproducible, and inconsistent.

On the other hand, the use of energetic phase change materials (such as magnesium-iron-salt granules and phase change material in various proportions), enables brief, moderated, controllable heating steps with controlled maximum temperature and, if so desired, with temperature plateaus of several minutes, as illustrated in FIG. 12. Specifically, eluants from blood samples mixed with lysis buffer that were heated during nucleic acid isolation with a nucleic acid-binding membrane enable much greater downstream polymerase efficiency than in the absence of heating.

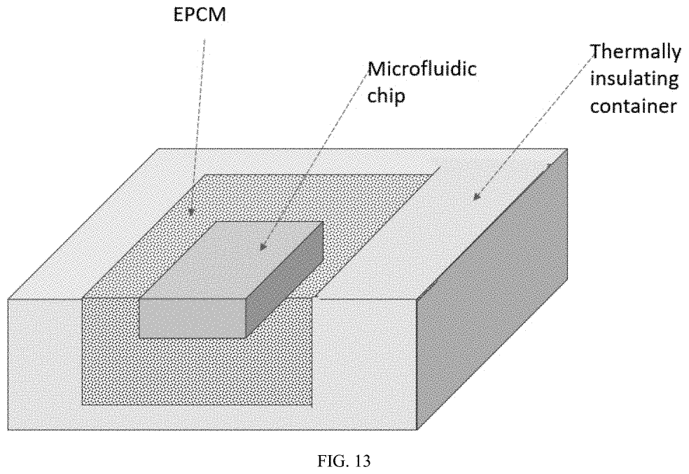

It should be understood that the controlled chemical heating with energetic phase change materials is also compatible with heating microfluidic devices (e.g., microfluidic chips, cartridges, or cassettes) as shown in FIG. 13. That figure provides an example device according to the present disclosure, which device includes a microfluidic chip in thermal communication with (and contacting) an EPCM, with thermally insulating container disposed around the EPCM. In this arrangement, the top surface of the microfluidic chip is exposed for fluidic exchange and optical excitation and interrogation. The configuration shown can accommodate modifications, e.g., including water pouches and/or capillary channels to distribute water to initiate and sustain the exothermic reaction.

Aspects

The following Aspects are illustrative only and do not limit the scope of the present disclosure or the appended claims.

Aspect 1. An exothermic composite, comprising:

a reactive material (RM) that undergoes an exothermic reaction upon contact with an oxidizer, and

a phase-changing thermal storage material (PCM) having a phase change temperature, wherein

(1) RM and PCM are intermixed with one another or

(2) one of RM and PCM is interpenetrated with the other.

Aspect 2. The composite of Aspect 1, wherein RM comprises a metal or a metal alloy.

Aspect 3. The composite of Aspect 1, wherein RM comprises a magnesium-iron alloy, calcium oxide, sodium acetate, potassium permanganate, iron, lithium, or any combination thereof.

Aspect 4. The composite of any one of Aspects 1-3, wherein PCM comprises wax, a thermoplastic, a salt hydrate, a fatty acid, a fatty acid ester, or any combination thereof.

Aspect 5. composite of any one of Aspects 1-4, wherein the composite, following contact with sufficient oxidizer, maintains for a time interval a substantially isothermal temperature TI1 that is sufficient to support a selected biological, physical, or chemical process.

Aspect 6. The composite of Aspect 5, wherein TI1 is from about 35 deg. C. to about 95 deg. C.

Aspect 7. The composite of any one of Aspects 1-6, wherein RM is denoted RMA, wherein PCM is denoted PCMA, wherein RMA and PCMA define a composite material ECA, and wherein the composite comprises an additional phase-changing thermal storage material (PCMB) that has a phase change temperature that differs from the phase transition temperature of PCMA.

Aspect 8. The composite of Aspect 7, wherein PCMB is at least partially enclosed within ECA.

Aspect 9. The composite of any one of Aspects 7-8, further comprising an additional reactive material RMB, and wherein (1) RMB and PCMB are intermixed with one another to form a composite ECB or (2) one of RMB and PCMB is interpenetrated with the other to form the composite ECB.

Aspect 10. The composite of Aspect 9, wherein ECB is at least partially enclosed within ECA or ECA is at least partially enclosed within ECB.

Aspect 11. The composite of any one of Aspects 7-10, wherein PCMB has a phase change temperature in the range of from about 35 deg. C. to about 45 deg. C, and wherein PCMA has a phase change temperature in the range of from about 55 deg. C. and about 75 deg. C.

Aspect 12. The composite of any one of Aspects 7-11, wherein the composite, following contact with sufficient oxidizer, maintains for a time interval a substantially isothermal temperature TI2 that is sufficient to support a selected biological or chemical process.

Aspect 13. The composite of Aspect 12, wherein TI2 is from about 35 deg.

C. to about 98 deg. C,

Aspect 14. The composite of any one of Aspects 1-13, wherein the oxidizer is water, oxygen, or an organic liquid.

Aspect 15. The composite of any one of Aspects 1-14, further comprising a removeable seal, conduit, or porous structure that separates RM from the oxidizer.

Aspect 16. The composite of any one of Aspects 1-15, wherein the exothermic reaction is initiated by opening a valve, imbibing water, removing a seal, or adding water.

Aspect 17. A device, comprising (1) a sample container that defines a sample volume therein or (2) a receptacle configured to accept a sample container defining a sample volume therein, and

the device configured such that the sample container is in thermal communication with a composite according to any one of Aspects 1-16.

A sample container can be, e.g., a tube, a vial, a microfluidic chip, a cartridge, a cassette, a paper-based reaction, a syringe, and the like. As described, a device can incorporate the sample container therein. A device can also, as described, include a receptacle to accept a sample container.

A device (and/or a sample container associated with the device) can include an amount of EPCM (e.g., a composition according to any one of Aspects 1-16) on or in the sample container. A device (and/or a sample container associated with the device) can include an amount of oxidizer (e.g., water, buffer) in the sample container, e.g., present in a pouch, blister, or other storage volume.

An example such device is shown in FIG. 13. As shown in that figure, a microfluidic chip can be in thermal communication (e.g., directly contacting) with an EPCM material. The EPCM material can be disposed within a thermally insulating container, which thermally insulating container can allow for removal and/or replacement of the EPCM and any containers (e.g., the microfluidic chip shown in FIG. 13) that are engaged with the device. In this way, one can provide devices as kits, with the kits including a thermally insulating container that accommodates replaceable EPCMs and microfluidic devices. One can provide such kits in a modular fashion, e.g., a single thermally insulating container that accommodates various combinations of EPCM and microfluidic chips. For example, a first EPCM-microfluidic chip combination can be configured to support a certain polymerase amplification reaction, whereas a second EPCM-microfluidic chip combination (which can also be accommodated by the same thermally insulating container that accommodates the first EPCM-microfluidic chip combination) may support a different incubation reaction such as polymerase amplification or nucleic acid isolation from coarse sample such that one or both of the second EPCM and second microfluidic chip differ from the first EPCM and first microfluidic chip.

The EPCM need not be external to the microfluidic chip or cassette. In some instances, the EPCM can be incorporated on or even in the microfluidic device or cassette.

As shown, a microfluidic chip can be available to have samples introduced and/or withdrawn while the microfluidic chip is engaged with the EPCM. A lid or other hatch can enclose at least part of the microfluidic chip. A device can include on-board water sources (e.g., pouches) or other materials besides water that activate the EPCM. In this way, device can be useful in the field and/or at the point of care/need, where access to electricity and other utilities may be limited or even impossible. A device can include (not shown in FIG. 13) capillaries or other channels configured to deliver water or other material to the EPCM so as to activate the EPCM's heating. In lieu of water, the oxidizer may comprise waste solutions such as discharged lysis buffer or water-based wash solutions. A device can include (also not shown in FIG. 13) thermometers or other monitors configured to monitor a condition (e.g., a temperature, an elapsed time) of the device. A device can, of course, include multiple EPCMs and/or multiple microfluidic elements. As one example, an EPCM can engage with two separate microfluidic elements. A given container can contain therein multiple EPCMs and/or multiple microfluidic elements.

Aspect 18. The device of Aspect 17, wherein the device is configured such that following contact with sufficient oxidizer, a sample volume within the sample container maintains for a time interval a substantially isothermal temperature TI1 that is sufficient to support a selected biological or chemical process.

Aspect 19. The device of Aspect 18, wherein TI1 is from about 35 deg. C. to about 98 deg. C

Aspect 20. The device of any one of Aspects 18-19, wherein the time interval is for from about 5 minutes to about 60 minutes.

Aspect 21. The device of any one of Aspects 18-20, wherein the device comprises a composite according to any one of Aspects 7-13 and wherein the device is configured such that following contact with sufficient oxidizer, a sample volume within the sample container maintains for an additional time interval a substantially isothermal temperature TI2 that is sufficient to support a selected biological or chemical process.

Aspect 22. The device of Aspect 21, wherein TI2 is from about 35 deg. C. to about 98 deg. C, and wherein TI2 is optionally higher than TI1.

Aspect 23. The device of any one of Aspects 21-22, wherein the device is configured such that the sample container contacts PCMB or such that the sample container is closer to PCMB than to PCMA.

Aspect 24. The device of any one of Aspects 21-23, wherein the additional time interval is for from about 10 minutes to about 60 minutes.

As one non-limiting example, a device can be configured such that TI1 is maintained at from about 35 to about 45 deg. C for from about 5 to about 20 minutes and that TI2 is maintained at from about 55 to about 75 deg. C. for from about 10 to about 60 minutes.

Aspect 25. The device of any one of Aspects 17-24, further comprising an insulating portion that at least partially encloses the composite.

Aspect 26. The device of any one of Aspects 17-25, further comprising a partition that separates the composite from the oxidizer.

Aspect 27. The device of any one of Aspects 17-26, further comprising a conduit placing the composite into fluid communication with a reservoir configured to contain a liquid, the conduit optionally capable of transporting the liquid by capillary action.

Aspect 28. The device of any one of Aspects 17-27, wherein the sample container is configured as an enzymatic amplification chamber.

Aspect 29. The device of any one of Aspects 17-28, further comprising a thermally-actuated fluidic element, the thermally-actuated fluidic element being in thermal communication with the composite. Such an element can be, e.g., a valve, a reagent (e.g., a reagent stored within a thermally-sensitive encapsulant), and the like.

Aspect 30. The device of any one of Aspects 17-29, further comprising an imager and/or an illumination source, the illumination source optionally configured to excite a fluorescent reporter within the sample container.

Aspect 31. A method, comprising the use of a composite according to claim 1.

Aspect 32. A method, comprising the use of a device according to Aspect 17.

Aspect 33. The method of Aspect 31, wherein the use comprises incubation of nucleic acid amplification.

Aspect 34. The method of claim 31, wherein the use comprises control of a heating rate and a temperature maximum.

Aspect 35. A method, comprising:

contacting, with an oxidizer, an exothermic composite according to any one of Aspects 1-12, the contacting giving rise to a sample container that is in thermal communication with the composite maintaining a temperature of about the phase change temperature of PCMA, and

effecting a biological or chemical interaction in the sample container.

Aspect 36. The method of Aspect 35, wherein the biological or chemical interaction is nucleic acid amplification.

Aspect 37. The method of any one of Aspects 35-36, wherein the sample container maintains, for from 5 to about 60 minutes, a temperature of between 35 and about 45 deg. C.

Aspect 38. The method of any one of Aspects 35-36, wherein the sample container maintains, for from 10 to about 60 minutes, a temperature of between 55 and about 70 deg. C.

Aspect 39. The method of any one of Aspects 35-38, wherein the contacting gives rise to the sample container in thermal communication with the composite heating material maintaining a first non-ambient temperature during a first time interval and maintaining a second non-ambient temperature during a second time interval.

Aspect 40. The method of Aspect 39, wherein the first non-ambient temperature is from between 35 and about 45 deg. C. and the first time interval is from about 5 to about 20 minutes.

Aspect 41. The method of any one of Aspects 39-40, wherein second non-ambient temperature is between 55 and about 70 deg. C. and the second time interval is from about 10 to about 60 minutes.

Aspect 42. The method of any one of Aspects 39-41, further comprising detecting a product of nucleic acid amplification.

Aspect 43. The method of Aspect 42, wherein the detecting comprises visual detection, fluorescent detection, electrochemical detection, or any combination thereof.

Aspect 44. The method of any one of Aspects 42-43, further comprising illuminating the product.

Aspect 45. A garment, shelter, blanket, or container comprising a composite according to any one of Aspects 1-16.

What is claimed:

1. An exothermic composite, comprising:
a reactive material (RM) that undergoes an exothermic reaction upon contact with an oxidizer, and
a phase-changing thermal storage material (PCM) having a phase change temperature, wherein
(1) RM and PCM are intermixed with one another or
(2) one of RM and PCM is interpenetrated with the other.

2. The composite of claim 1, wherein RM comprises a metal or a metal alloy.

3. The composite of claim 1, wherein RM comprises a magnesium-iron alloy, calcium oxide, sodium acetate, potassium permanganate, iron, lithium, or any combination thereof.

4. The composite of claim 1, wherein PCM comprises wax, a thermoplastic, a salt hydrate, a fatty acid, a fatty acid ester, or any combination thereof.

5. The composite of claim 1, wherein the composite, following contact with sufficient oxidizer, maintains for a time interval a substantially isothermal temperature TI1 that is sufficient to support a selected biological, physical, or chemical process.

6. The composite of claim 5, wherein TI1 is from about 35 deg. C. to about 95 deg. C.

7. The composite of claim 1, wherein RM is denoted RMA, wherein PCM is denoted PCMA, wherein RMA and PCMA define a composite material ECA, and wherein the composite comprises an additional phase-changing thermal storage material (PCMB) that has a phase change temperature that differs from the phase transition temperature of PCMA.

8. The composite of claim 7, wherein PCMB is at least partially enclosed within ECA.

9. The composite of claim 7, further comprising an additional reactive material RMB, and wherein (1) RMB and PCMB are intermixed with one another to form a composite ECB or (2) one of RMB and PCMB is interpenetrated with the other to form the composite ECB.

10. The composite of claim 9, wherein ECB is at least partially enclosed within ECA or ECA is at least partially enclosed within ECB.

11. The composite of claim 7, wherein PCMB has a phase change temperature in the range of from about 35 deg. C. to about 45 deg. C, and wherein PCMA has a phase change temperature in the range of from about 55 deg. C. and about 75 deg. C.

12. The composite of claim 7, wherein the composite, following contact with sufficient oxidizer, maintains for a time interval a substantially isothermal temperature T12 that is sufficient to support a selected biological or chemical process.

13. The composite of claim 12, wherein T12 is from about 35 deg. C. to about 98 deg. C.

14. The composite of claim 1, wherein the oxidizer is water, oxygen, or an organic liquid.

15. The composite of claim 1, further comprising a removeable seal, conduit, or porous structure that separates RM from the oxidizer.

16. The composite of claim 1, wherein the exothermic reaction is initiated by opening a valve, removing a seal, imbibing water, or adding water.

17. A device, comprising (1) a sample container that defines a sample volume therein or (2) a receptacle configured to accept a sample container defining a sample volume therein, and
the device configured such that the sample container is in thermal communication with a composite according to claim 1.

18. The device of claim 17, wherein the device is configured such that following contact with sufficient oxidizer, a sample volume within the sample container maintains for a time interval a substantially isothermal temperature TI1 that is sufficient to support a selected biological or chemical process.

19. The device of claim 18, wherein TI1 is from about 35 deg. C. to about 98 deg. C.

20. The device of claim 18, wherein the time interval is for from about 5 minutes to about 60 minutes.

21. The device of claim 18, wherein the device is configured such that following contact with sufficient oxidizer, a sample volume within the sample container maintains for an additional time interval a substantially isothermal temperature TI2 that is sufficient to support a selected biological or chemical process.

22. The device of claim 21, wherein TI2 is from about 35 deg. C. to about 98 deg. C, and wherein TI2 is optionally higher than TI1.

23. The device of claim 21, wherein the device is configured such that the sample container contacts PCMB or such that the sample container is closer to PCMB than to PCMA.

24. The device of claim 21, wherein the additional time interval is for from about 10 minutes to about 60 minutes.

25. The device of claim 17, further comprising an insulating portion that at least partially encloses the composite.

26. The device of claim 17, further comprising a partition that separates the composite from the oxidizer.

27. The device of claim 17, further comprising a conduit placing the composite into fluid communication with a reservoir configured to contain a liquid, the conduit optionally capable of transporting the liquid by capillary action.

28. The device of claim 17, wherein the sample container is configured as an enzymatic amplification chamber.

29. The device of claim 17, further comprising a thermally-actuated fluidic element, the thermally-actuated fluidic element being in thermal communication with the composite.

30. The device of claim 17, further comprising an imager and/or an illumination source, the illumination source optionally configured to excite a fluorescent reporter within the sample container.

31. A method, comprising the use of a composite according to claim 1.

32. A method, comprising the use of a device according to claim 17.

33. The method of claim 31, wherein the use comprises incubation of nucleic acid amplification.

34. The method of claim 31, wherein the use comprises control of a heating rate and a temperature maximum.

35. A method, comprising:
contacting, with an oxidizer, an exothermic composite according to claim 1, the contacting giving rise to a sample container that is in thermal communication with the composite maintaining a temperature of about the phase change temperature of PCMA, and
effecting a biological or chemical interaction in the sample container.

36. The method of claim 35, wherein the biological or chemical interaction is nucleic acid amplification.

37. The method of claim 35, wherein the sample container maintains, for from 5 to about 60 minutes, a temperature of between 35 and about 45 deg. C.

38. The method of claim 35, wherein the sample container maintains, for from 10 to about 60 minutes, a temperature of between 55 and about 70 deg. C.

39. The method of claim 35, wherein the contacting gives rise to the sample container in thermal communication with the composite heating material maintaining a first non-ambient temperature during a first time interval and maintaining a second non-ambient temperature during a second time interval.

40. The method of claim 39, wherein the first non-ambient temperature is from between 35 and about 45 deg. C. and the first time interval is from about 5 to about 20 minutes.

41. The method of claim 39, wherein second non-ambient temperature is between 55 and about 70 deg. C. and the second time interval is from about 10 to about 60 minutes.

42. The method of claim 39, further comprising detecting a product of nucleic acid amplification.

43. The method of claim 42, wherein the detecting comprises visual detection, fluorescent detection, electrochemical detection, or any combination thereof.

44. The method of claim 42, further comprising illuminating the product.

45. A garment, shelter, blanket, or container comprising a composite according to claim 1.
